(19) United States
(12) Patent Application Publication
(10) Pub. No.: US 2019/0078146 A1
(43) Pub. Date: Mar. 14, 2019

(54) SYSTEM AND METHOD FOR GENE DETECTION

(71) Applicant: Lanfang XIAN, Hong Kong (HK)

(72) Inventor: Lanfang XIAN, Hong Kong (HK)

(73) Assignee: XIANLAN TECHNOLOGY CO., LIMITED, Hong Kong (HK)

(21) Appl. No.: 16/191,449

(22) Filed: Nov. 15, 2018

(51) Int. Cl. C12Q 1/6816; H03F 3/45; G01R 33/12; G01R 33/00

(52) U.S. Cl. CPC C12Q 1/6816; H03F 3/45; H03F 2203/45598; G01R 33/0029; G01R 33/1269

(57) ABSTRACT

A system for gene detection includes a sensor module including a plurality of magnetic sensors in an arrangement of a matrix and a signal processing chip including a front-end circuit and a signal processing circuit. The sensor module is formed on the signal processing chip through sputtering. The signal processing chip is configured to transform variation of the reluctivity of the magnetic sensors into a first electrical signal, process the first electrical signal and output a second electrical signal representing a detection result of a DNA molecule to be detected. The front-end circuit includes a row address selector, a column address selector, a pre-amplifier and a biasing circuit. The magnetic sensors are chemically pretreated and combined with a biological probe. The combined magnetic sensors and biological probe are in sufficient contact with combined magnetic particles and DNA molecules to be detected.

### FIELD OF THE PATENT APPLICATION

The present patent application generally relates to medical electronics and more specifically to a system and a method for gene detection.

### BACKGROUND

Gene or molecular biology detection is important to early diagnosis of diseases. Conventional gene detection depends on optical means which may lead to optical losses such as reflection and refraction, and therefore the resolution of the detection is relatively low and the detection is expensive and needs to be operated by professional staff. In recent years, gene detection systems based on magnetic labels have been proposed and such systems are more stable, faster and easier to operate compared with conventional gene detection systems. However, sensitivity, power consumption and yield are still the main bottlenecks of these systems.

### SUMMARY

The present patent application is directed to a system and method for gene detection. In one aspect, the system for gene detection includes a sensor module including a plurality of magnetic sensors in an arrangement of a matrix; and a signal processing chip configured to transform variation of the reluctivity of the magnetic sensors into a first electrical signal, process the first electrical signal and output a second electrical signal representing a detection result of a DNA molecule to be detected. The plurality of magnetic sensors are chemically pretreated and combined with a biological probe. The combined magnetic sensors and biological probe are disposed in a DC magnetic field and an AC magnetic field, and in sufficient contact with combined magnetic particles and DNA molecules to be detected, so that the DNA molecules to be detected are matched and hybridized with the biological probe. The plurality of magnetic sensors are then cleaned so that the DNA molecules to be detected that are not hybridized are removed; and the magnetic particles combined with the DNA molecules to be detected that are hybridized are relatively fixed above the magnetic sensors so that a scattered magnetic field is formed and the reluctivity of the magnetic sensors varies under the scattered magnetic field.

The sensor module may be formed on the signal processing chip through sputtering. The signal processing chip may include a front-end circuit configured to transform variation of the reluctivity of the magnetic sensors into a first electrical signal and a signal processing circuit configured to process the first electrical signal and output a second electrical signal representing a detection result of a DNA molecule to be detected.

The front-end circuit may include a row address selector and a column address selector coordinated with each other and configured to allow the current to flow into selected magnetic sensors.

The front-end circuit further may include a pre-amplifier configured to amplify the electrical signal representing variation of the reluctivity of the magnetic sensors so as to produce the first electrical signal.

The pre-amplifier may include a differential amplifier, a first clipper stabilizing circuit, a second clipper stabilizing circuit, a third clipper stabilizing circuit, a first resistor and a second resistor; the differential amplifier may include a positive input port, a negative input port, a first positive output port, a first negative output port, a second positive output port and a second negative output port; the first clipper stabilizing circuit is connected with the first positive output port and the first negative output port; the second clipper stabilizing circuit is connected with the second positive output port and the second negative output port; the third clipper stabilizing circuit is connected with the positive input port and the negative input port; the positive input port is connected with the first negative output port through the first resistor; the negative input port is connected with the second negative output port through the second resistor; the input signal of the differential amplifier is an electrical signal output from the magnetic sensors and representing variation of the reluctivity of the magnetic sensors, and input through the positive input port and the negative input port; the output signals of the differential amplifier may include a first output signal output from the first positive output port and a second output signal output from the second positive output port.

The pre-amplifier may further include a third resistor, a fourth resistor, a first capacitor and a second capacitor; the positive input port is connected with the ground through the third resistor and the first capacitor; the second negative output port is connected with the ground through the fourth resistor and the second capacitor.

The front-end circuit may further include a biasing circuit; the biasing circuit may include a plurality of diodes individually corresponding to the plurality of magnetic sensors; each diode is connected in series with a corresponding magnetic sensor so as to prevent the current from flowing into unselected magnetic sensors.

In another aspect, the present patent application provides a system for gene detection. The system for gene detection includes a sensor module including a plurality of magnetic sensors in an arrangement of a matrix; and a signal processing chip. The plurality of magnetic sensors are chemically pretreated and combined with a biological probe. The combined magnetic sensors and biological probe are disposed in a DC magnetic field and an AC magnetic field, and in sufficient contact with combined magnetic particles and DNA molecules to be detected, so that the DNA molecules to be detected are matched and hybridized with the biological probe. The plurality of magnetic sensors are then cleaned so that the DNA molecules to be detected that are not hybridized are removed. The magnetic particles combined with the DNA molecules to be detected that are hybridized are relatively fixed above the magnetic sensors so that a scattered magnetic field is formed and the reluctivity of the magnetic sensors varies under the scattered magnetic field. The sensor module is formed on the signal processing chip through sputtering. The signal processing chip includes a front-end circuit configured to transform variation of the reluctivity of the magnetic sensors into a first electrical signal and a signal processing circuit configured to process the first electrical signal and output a second electrical signal representing a detection result of a DNA molecule to be detected. The front-end circuit includes a row address selector and a column address selector coordinated with each other and configured to allow the current to flow into selected magnetic sensors, and a biasing circuit and a pre-amplifier. The biasing circuit includes a plurality of diodes individually corresponding to the plurality of magnetic sensors; each diode is connected in series with a corresponding magnetic sensor so as to prevent the current from flowing into unselected magnetic sensors. The pre-amplifier includes a differential amplifier, a first clipper stabilizing circuit, a second clipper stabilizing circuit, a third clipper stabilizing circuit, a first resistor and a second resistor; the differential amplifier includes a positive input port, a negative input port, a first positive output port, a first negative output port, a second positive output port and a second negative output port. The first clipper stabilizing circuit is connected with the first positive output port and the first negative output port. The second clipper stabilizing circuit is connected with the second positive output port and the second negative output port. The third clipper stabilizing circuit is connected with the positive input port and the negative input port. The positive input port is connected with the first negative output port through the first resistor. The negative input port is connected with the second negative output port through the second resistor. The input signal of the differential amplifier is an electrical signal output from the plurality of magnetic sensors and representing variation of the reluctivity of the magnetic sensors, and input through the positive input port and the negative input port; and the output signals of the differential amplifier include a first output signal output from the first positive output port and a second output signal output from the second positive output port.

The pre-amplifier may further include a third resistor, a fourth resistor, a first capacitor and a second capacitor; the positive input port is connected to the ground through the third resistor and the first capacitor; the second negative output port is connected with the ground through the fourth resistor and the second capacitor.

In yet another aspect, the present patent application provides a method for gene detection. The method for gene detection includes step 1: magnetizing a plurality of magnetic particles with a DC magnetic field and an AC magnetic field; step 2: combining a plurality of magnetized magnetic particles and a plurality of DNA molecules to be detected; step 3: chemically pretreating magnetic sensors and combining the magnetic sensors with a biological probe; step 4: establishing the DC magnetic field and the AC magnetic field around the combined magnetic sensors and biological probe as described in step 3 and establishing a signal baseline; step 5: disposing the combined DNA molecules to be detected and magnetic particles as described in step 2 on the combined magnetic sensors and biological probe as described in step 3 so that the combined DNA molecules to be detected and magnetic particles are in sufficient contact with the combined magnetic sensors and biological probe; step 6: cleaning the plurality of magnetic sensors, removing the DNA molecules to be detected that are not hybridized, and then relatively fixing the magnetic particles combined with the hybridized DNA molecules to be detected above the magnetic sensors so that a scattered magnetic field can be formed; step 7: transforming variation of the reluctivity of the plurality of magnetic sensors into a first electrical signal through a signal processing chip, the reluctivity of the plurality of magnetic sensors varying under the scattered magnetic field; and step 8: processing the first electrical signal and outputting a second electrical signal representing a detection result of the DNA molecules to be detected with the signal processing chip.

The sensor module may be formed on the signal processing chip through sputtering. The signal processing chip may include a front-end circuit configured to transform variation of the reluctivity of the magnetic sensors into a first electrical signal and a signal processing circuit configured to process the first electrical signal and output a second electrical signal representing a detection result of a DNA molecule to be detected.

The front-end circuit may include a row address selector and a column address selector coordinated with each other and configured to allow the current to flow into selected magnetic sensors.

The front-end circuit may further include a pre-amplifier configured to amplify the electrical signal representing variation of the reluctivity of the magnetic sensors so as to produce the first electrical signal.

The pre-amplifier may include a differential amplifier, a first clipper stabilizing circuit, a second clipper stabilizing circuit, a third clipper stabilizing circuit, a first resistor and a second resistor; the differential amplifier may include a positive input port, a negative input port, a first positive output port, a first negative output port, a second positive output port and a second negative output port; the first clipper stabilizing circuit is connected with the first positive output port and the first negative output port; the second clipper stabilizing circuit is connected with the second positive output port and the second negative output port; the third clipper stabilizing circuit is connected with the positive input port and the negative input port; the positive input port is connected with the first negative output port through the first resistor; the negative input port is connected with the second negative output port through the second resistor; the input signal of the differential amplifier is an electrical signal output from the magnetic sensors and representing variation of the reluctivity of the magnetic sensors, and input through the positive input port and the negative input port; the output signals of the differential amplifier may include a first output signal output from the first positive output port and a second output signal output from the second positive output port.

The pre-amplifier may further include a third resistor, a fourth resistor, a first capacitor and a second capacitor; the positive input port is connected with the ground through the third resistor and the first capacitor; the second negative output port is connected with the ground through the fourth resistor and the second capacitor.

The front-end circuit may further include a biasing circuit; the biasing circuit may include a plurality of diodes individually corresponding to the plurality of magnetic sensors; each diode is connected in series with a corresponding magnetic sensor so as to prevent the current from flowing into unselected magnetic sensors.

### BRIEF DESCRIPTION OF THE DRAWINGS

FIG. 1 is a block diagram of a system for gene detection in accordance with an embodiment of the present patent application.

FIG. 2 illustrates the structure of a magnetic sensor matrix of the system for gene detection as depicted in FIG. 1.

FIG. 3 illustrates the structure of a magnetic sensor of the magnetic sensor matrix as depicted in FIG. 2.

FIG. 4 is a block diagram of a signal processing chip of the system for gene detection as depicted in FIG. 1.

FIG. 5 is a schematic circuit diagram of a front-end circuit of the system for gene detection as depicted in FIG. 1.

FIG. 6 is a schematic circuit diagram of a pre-amplifier of the system for gene detection as depicted in FIG. 1.

FIG. 7 is a flowchart illustrating a method for gene detection executed by the system as depicted in FIG. 1.

### DETAILED DESCRIPTION

Reference will now be made in detail to a preferred embodiment of the system and method for gene detection disclosed in the present patent application, examples of which are also provided in the following description. Exemplary embodiments of the system and method for gene detection disclosed in the present patent application are described in detail, although it will be apparent to those skilled in the relevant art that some features that are not particularly important to an understanding of the system and method for gene detection may not be shown for the sake of clarity.

Furthermore, it should be understood that the system and method for gene detection disclosed in the present patent application is not limited to the precise embodiments described below and that various changes and modifications thereof may be effected by one skilled in the art without departing from the spirit or scope of the protection. For example, elements and/or features of different illustrative embodiments may be combined with each other and/or substituted for each other within the scope of this disclosure.

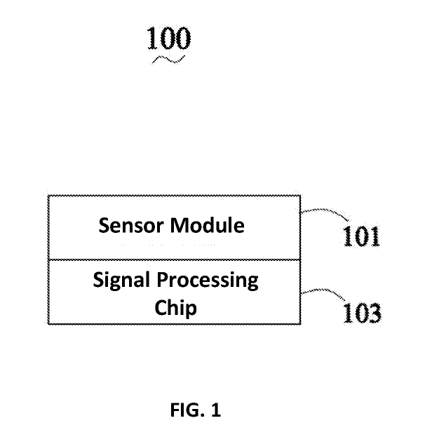

FIG. 1 is a block diagram of a system for gene detection in accordance with an embodiment of the present patent application. Referring to FIG. 1, the system for gene detection 100 includes a sensor module 101 and a signal processing chip 103.

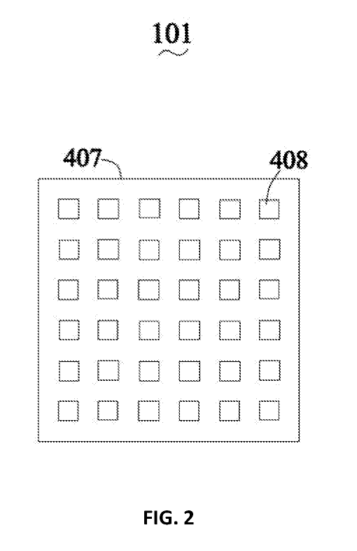

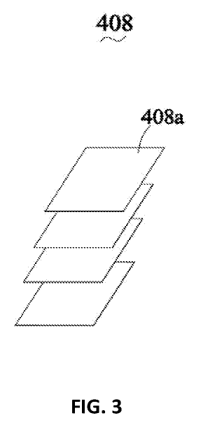

Referring to FIG. 2 and FIG. 3, the sensor module 101 is formed on the signal processing chip 103 through sputtering and the sensor module 101 includes a magnetic sensor matrix 407. The magnetic sensor matrix 407 includes a number of magnetic sensors 408 in an arrangement of a matrix. Each magnetic sensor 408 is formed by stacking layers of magnetic materials 408a, and the reluctivity of each magnetic sensor varies with the spin alignment of the electrons of two layers of magnetic materials 408a. When disposed in an external magnetic field, the reluctivity of the stacked magnetic materials 408a varies with the intensity of the external magnetic field.

The multiple magnetic sensors 408 are chemically pretreated, so that the multiple magnetic sensors 408 are combined with a biological probe (not shown in the figures). The combined magnetic sensors 408 and biological probe are disposed in a DC magnetic field and an AC magnetic field and in sufficient contact with the combined multiple magnetic particles and DNA molecules to be detected, so that the DNA molecules to be detected are matched and hybridized with the biological probe. The multiple magnetic sensors 408 are then cleaned, and the DNA molecules to be detected that are not hybridized are removed. The magnetic particles combined with the DNA molecules that are hybridized are relatively fixed above the magnetic sensors 408, so that a scattered magnetic field can be formed. The reluctivity of the multiple magnetic sensors 408 varies under the scattered magnetic field.

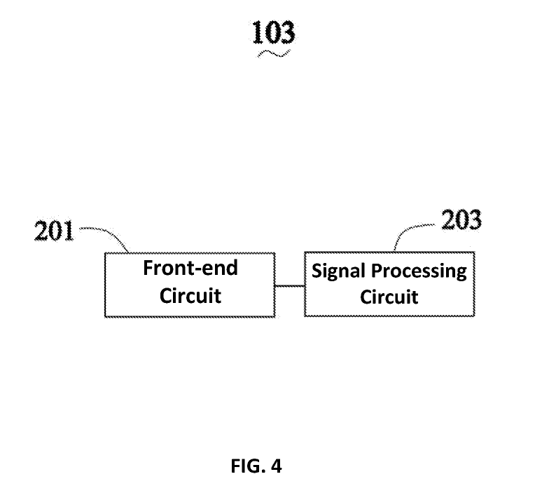

Referring to FIG. 4, the signal processing chip 103 includes a front-end circuit 201 and a signal processing circuit 203. The front-end circuit 201 is configured to transform variation of the reluctivity of the multiple magnetic sensors 408 in the sensor module 101 into a first electrical signal (e.g. a voltage signal Vin). The signal processing circuit 203 is configured to process the electrical signal and output a second electrical signal representing a detection result of the DNA molecules to be detected (e.g. a voltage signal Vout).

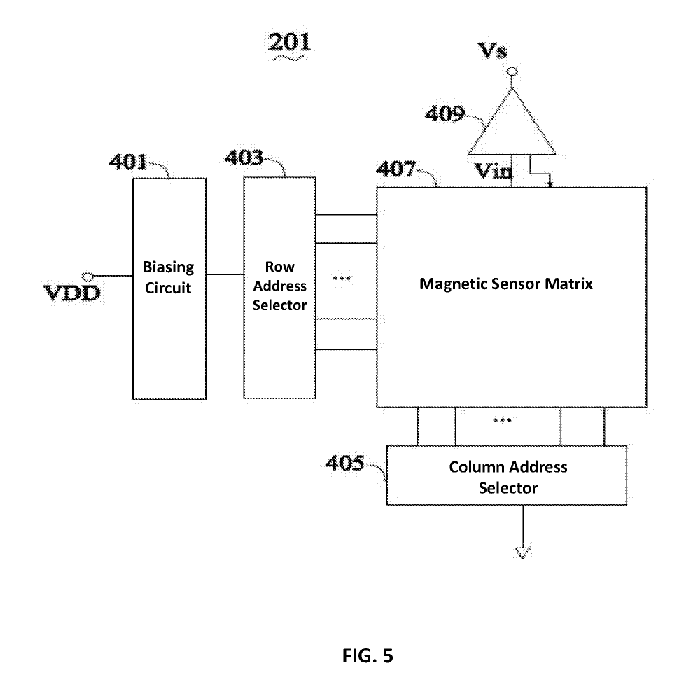

Referring to FIG. 5, the front-end circuit 201 includes a row address selector 403, a column address selector 405, a biasing circuit 401 and a pre-amplifier 409.

The row address selector 403 includes multiple row switches and the column address selector 405 includes multiple column switches. The multiple row switches and the multiple column switches are coordinated with each other, connected with an external power supply VDD, and configured to allow the current to flow into selected magnetic sensors 408.

The biasing circuit 401 includes multiple diodes. The multiple diodes are individually corresponding to the multiple magnetic sensors 408 and each diode is connected in series with a corresponding magnetic sensor 408 so as to prevent the current from flowing into unselected magnetic sensors 408. The configuration of the multiple diodes helps to improve the accuracy of the system for gene detection 100 and reduce power consumption.

Because one magnetic sensor 408 is selected at a time, all magnetic sensors 408 can share the same biasing circuit 401 and the same pre-amplifier 409, which reduces power consumption and system noise. At the same time, the noise of the magnetic sensors 408 is at the same order of magnitude as the smallest detection signal and the noise of the magnetic sensors 408 is determined by its own material and structure, and therefore the noise of the front-end circuit 201 is lower than that of the magnetic sensors 408.

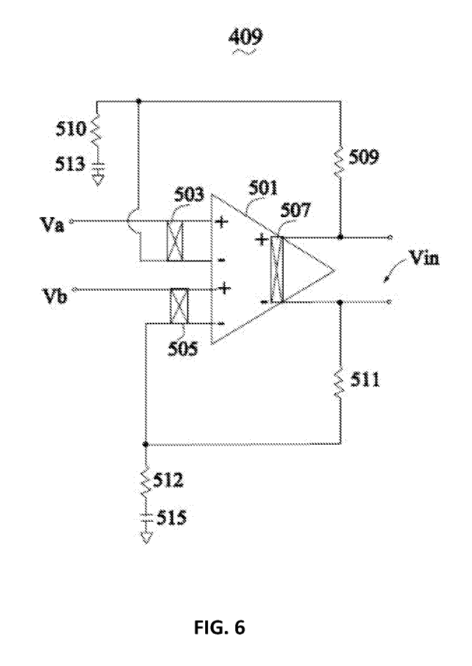

Referring to FIG. 6, the pre-amplifier 409 is configured to amplify the electrical signal representing variation of the reluctivity of the magnetic sensors 408 so as to produce the first electrical signal and the pre-amplifier 409 includes a differential amplifier 501, a first clipper stabilizing circuit 503, a second clipper stabilizing circuit 505, a third clipper stabilizing circuit 507, a first resistor 509, a second resistor 511, a third resistor 510, a fourth resistor 512, a first capacitor 513 and a second capacitor 515.

The differential amplifier 501 includes a positive input port, a negative input port, a first positive output port, a first negative output port, a second positive output port, and a second negative output port.

The input signal Vin of the differential amplifier 501 is an electrical signal (e.g. AC signal) output from the multiple magnetic sensors 408, representing variation of the reluctivity of the multiple magnetic sensors 408, and input into the differential amplifier 501 through the positive input port and the negative input port. The output signal Vs of the differential amplifier 501 includes a first output signal Va and a second output signal Vb. The first output signal Va is output from the first positive output port. The second output signal Vb is output from the second positive output port. In this embodiment, the first output signal Va is an AC signal and the second output signal Vb is a DC signal.

The first clipper stabilizing circuit 503 is connected with the first positive output port and the first negative output port. The second clipper stabilizing circuit 505 is connected with the second positive output port and the second negative output port. The third clipper stabilizing circuit 507 is connected with the positive input port and the negative input port.

The positive input port is connected with the first negative output port through the first resistor 509. The negative input port is connected with the second negative output port through the second resistor 511. In addition, the positive input port is connected to the ground through the third resistor 510 and the first capacitor 513 and the second negative output port is connected to the ground through the fourth resistor 512 and the second capacitor 515 so as to filter stray waves and lower the noise of the output signal Vs.

The utilization of the first clipper stabilizing circuit 503, the second clipper stabilizing circuit 505 and the third clipper stabilizing circuit 507 lowers 1/f noise. The first capacitor 513 and the second capacitor 515 have effectively prevented DC current from flowing into a feedback loop and reduced the requirement for the drive capability of the output port. The input impedance of the pre-amplifier 409 is very high, so the input current is very small, which further suppress the 1/f noise. The pre-amplifier 409 realizes AC coupling and DC coupling while remaining low noise.

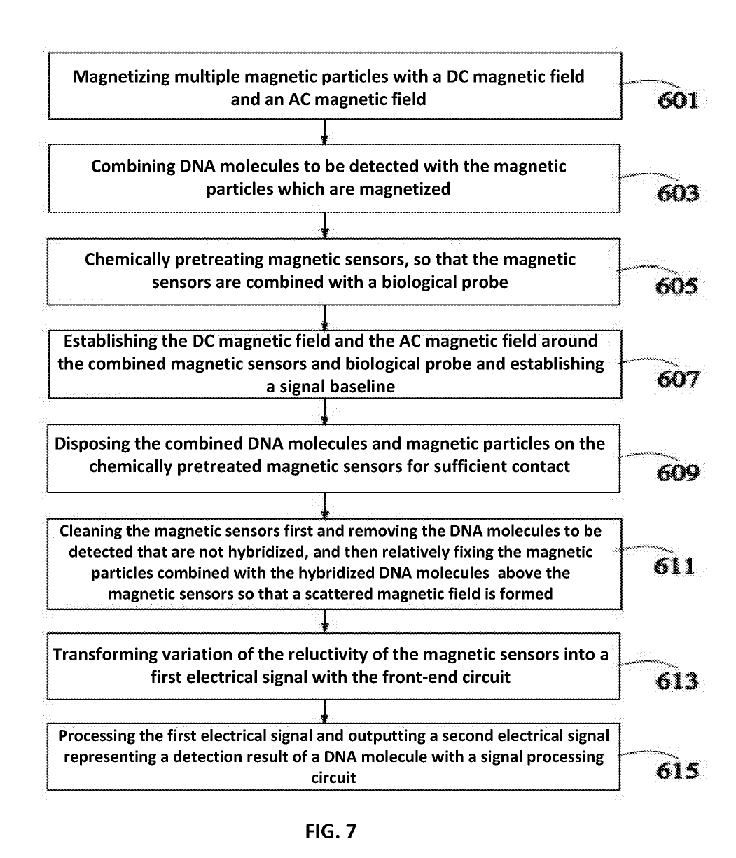

FIG. 7 is a flowchart illustrating a method for gene detection executed by the system as depicted in FIG. 1. The method includes the following steps:

Step 601: magnetizing multiple magnetic particles with a DC magnetic field and an AC magnetic field; the DC magnetic field and the AC magnetic field can be established through an electrified coil;

Step 603: combining DNA molecules to be detected with the multiple magnetic particles which are magnetized;

Step 605: chemically pretreating magnetic sensors, so that the magnetic sensors are combined with a biological probe;

Step 607: establishing the DC magnetic field and the AC magnetic field around the combined magnetic sensors and biological probe as described in the step 605, and establishing a signal baseline;

Step 609: disposing the combined DNA molecules and magnetic particles as described in the step 603 on the combined magnetic sensors and biological probe as described in the step 605 for sufficient contact;

Step 611: cleaning the magnetic sensors first and removing the DNA molecules to be detected that are not hybridized since the direct match of the DNA molecules to be detected and the biological probe will cause hybridization, and then relatively fixing the magnetic particles combined with the hybridized DNA molecules to be detected above the magnetic sensors so that a scattered magnetic field is formed;

Step 613: transforming variation of the reluctivity of the magnetic sensors into a first electrical signal with the front-end circuit 201, the reluctivity of the magnetic sensors varying under the scattered magnetic field; and

Step 615: processing the first electrical signal and outputting a second electrical signal representing a detection result of a DNA molecule to be detected with the signal processing circuit 203.

Compared with the conventional systems and methods for gene detection, the system and the method provided by the present patent application have the following advantages. (1) The signal processing chip 103 is formed on the sensor module 101 through sputtering, which contributes to good yield, high sensitivity, low parasitic capacitance, good scalability, smaller size of the system and stronger anti-interference capability (especially the capability of resisting electromagnetic interference). (2) All magnetic sensors share a biasing circuit and a pre-amplifier and clipper stabilizing circuits are used, which leads to high input impedance, small input current, and further suppressed 1/f noise, so that the noise of the signal processing chip 103 is lower than that of the sensor module and the sensitivity of the gene detection is improved. (3) Because all magnetic sensors share a biasing circuit and a pre-amplifier and the biasing circuit includes multiple diodes, the power consumption of the system for gene detection gets to be optimized. (4) Because the structure of the system for gene detection is simple, the production yield is high.

While the present patent application has been shown and described with particular references to a number of embodiments thereof, it should be noted that various other changes or modifications may be made without departing from the scope of the present invention.

What is claimed is:

1. A system for gene detection comprising:
a sensor module comprising a plurality of magnetic sensors in an arrangement of a matrix; and
a signal processing chip configured to transform variation of the reluctivity of the magnetic sensors into a first electrical signal, process the first electrical signal and output a second electrical signal representing a detection result of a DNA molecule to be detected; wherein:
the plurality of magnetic sensors are chemically pretreated and combined with a biological probe;
the combined magnetic sensors and biological probe are disposed in a DC magnetic field and an AC magnetic field, and in sufficient contact with combined magnetic particles and DNA molecules to be detected, so that the DNA molecules to be detected are matched and hybridized with the biological probe;
the plurality of magnetic sensors are then cleaned so that the DNA molecules to be detected that are not hybridized are removed; and
the magnetic particles combined with the DNA molecules to be detected that are hybridized are relatively fixed above the magnetic sensors so that a scattered magnetic field is formed and the reluctivity of the magnetic sensors varies under the scattered magnetic field.

2. The system for gene detection of claim 1, wherein the sensor module is formed on the signal processing chip through sputtering.

3. The system for gene detection of claim 1, wherein the signal processing chip comprises a front-end circuit configured to transform variation of the reluctivity of the magnetic sensors into a first electrical signal and a signal processing circuit configured to process the first electrical signal and output a second electrical signal representing a detection result of a DNA molecule to be detected.

4. The system for gene detection of claim 3, wherein the front-end circuit comprises a row address selector and a column address selector coordinated with each other and configured to allow the current to flow into selected magnetic sensors.

5. The system for gene detection of claim 4, wherein the front-end circuit further comprises a pre-amplifier configured to amplify the electrical signal representing variation of the reluctivity of the magnetic sensors so as to produce the first electrical signal.

6. The system for gene detection of claim 5, wherein the pre-amplifier comprises a differential amplifier, a first clipper stabilizing circuit, a second clipper stabilizing circuit, a third clipper stabilizing circuit, a first resistor and a second resistor; the differential amplifier comprises a positive input port, a negative input port, a first positive output port, a first negative output port, a second positive output port and a second negative output port; the first clipper stabilizing circuit is connected with the first positive output port and the first negative output port; the second clipper stabilizing circuit is connected with the second positive output port and the second negative output port; the third clipper stabilizing circuit is connected with the positive input port and the negative input port; the positive input port is connected with the first negative output port through the first resistor; the negative input port is connected with the second negative output port through the second resistor; the input signal of the differential amplifier is an electrical signal output from the magnetic sensors and representing variation of the reluctivity of the magnetic sensors, and input through the positive input port and the negative input port; the output signals of the differential amplifier comprise a first output signal output from the first positive output port and a second output signal output from the second positive output port.

7. The system for gene detection of claim 6, wherein the pre-amplifier further comprises a third resistor, a fourth resistor, a first capacitor and a second capacitor; the positive input port is connected with the ground through the third resistor and the first capacitor; the second negative output port is connected with the ground through the fourth resistor and the second capacitor.

8. The system for gene detection of claim 4, wherein the front-end circuit further comprises a biasing circuit; the biasing circuit comprises a plurality of diodes individually corresponding to the plurality of magnetic sensors; each diode is connected in series with a corresponding magnetic sensor so as to prevent the current from flowing into unselected magnetic sensors.

9. A system for gene detection comprising:
a sensor module comprising a plurality of magnetic sensors in an arrangement of a matrix; and
a signal processing chip; wherein:
the plurality of magnetic sensors are chemically pretreated and combined with a biological probe;
the combined magnetic sensors and biological probe are disposed in a DC magnetic field and an AC magnetic field, and in sufficient contact with combined magnetic particles and DNA molecules to be detected, so that the DNA molecules to be detected are matched and hybridized with the biological probe;
the plurality of magnetic sensors are then cleaned so that the DNA molecules to be detected that are not hybridized are removed;
the magnetic particles combined with the DNA molecules to be detected that are hybridized are relatively fixed above the magnetic sensors so that a scattered magnetic field is formed and the reluctivity of the magnetic sensors varies under the scattered magnetic field;
the sensor module is formed on the signal processing chip through sputtering;
the signal processing chip comprises a front-end circuit configured to transform variation of the reluctivity of the magnetic sensors into a first electrical signal and a signal processing circuit configured to process the first electrical signal and output a second electrical signal representing a detection result of a DNA molecule to be detected;
the front-end circuit comprises a row address selector and a column address selector coordinated with each other and configured to allow the current to flow into selected magnetic sensors, and a biasing circuit and a pre-amplifier;
the biasing circuit comprises a plurality of diodes individually corresponding to the plurality of magnetic sensors; each diode is connected in series with a corresponding magnetic sensor so as to prevent the current from flowing into unselected magnetic sensors;
the pre-amplifier comprises a differential amplifier, a first clipper stabilizing circuit, a second clipper stabilizing circuit, a third clipper stabilizing circuit, a first resistor and a second resistor; the differential amplifier comprises a positive input port, a negative input port, a first positive output port, a first negative output port, a second positive output port and a second negative output port;
the first clipper stabilizing circuit is connected with the first positive output port and the first negative output port;
the second clipper stabilizing circuit is connected with the second positive output port and the second negative output port;
the third clipper stabilizing circuit is connected with the positive input port and the negative input port;
the positive input port is connected with the first negative output port through the first resistor;
the negative input port is connected with the second negative output port through the second resistor;
the input signal of the differential amplifier is an electrical signal output from the plurality of magnetic sensors and representing variation of the reluctivity of the magnetic sensors, and input through the positive input port and the negative input port; and
the output signals of the differential amplifier comprise a first output signal output from the first positive output port and a second output signal output from the second positive output port.

10. The system for gene detection of claim 9, wherein the pre-amplifier further comprises a third resistor, a fourth resistor, a first capacitor and a second capacitor; the positive input port is connected to the ground through the third resistor and the first capacitor; the second negative output port is connected with the ground through the fourth resistor and the second capacitor.

11. A method for gene detection comprising:
step 1: magnetizing a plurality of magnetic particles with a DC magnetic field and an AC magnetic field;
step 2: combining a plurality of magnetized magnetic particles and a plurality of DNA molecules to be detected;
step 3: chemically pretreating magnetic sensors and combining the magnetic sensors with a biological probe;
step 4: establishing the DC magnetic field and the AC magnetic field around the combined magnetic sensors and biological probe as described in step 3 and establishing a signal baseline;
step 5: disposing the combined DNA molecules to be detected and magnetic particles as described in step 2 on the combined magnetic sensors and biological probe as described in step 3 so that the combined DNA molecules to be detected and magnetic particles are in sufficient contact with the combined magnetic sensors and biological probe;
step 6: cleaning the plurality of magnetic sensors, removing the DNA molecules to be detected that are not hybridized, and then relatively fixing the magnetic particles combined with the hybridized DNA molecules to be detected above the magnetic sensors so that a scattered magnetic field can be formed;
step 7: transforming variation of the reluctivity of the plurality of magnetic sensors into a first electrical signal through a signal processing chip, the reluctivity of the plurality of magnetic sensors varying under the scattered magnetic field; and
step 8: processing the first electrical signal and outputting a second electrical signal representing a detection result of the DNA molecules to be detected with the signal processing chip.

12. The method for gene detection of claim 11, wherein the sensor module is formed on the signal processing chip through sputtering.

13. The method for gene detection of claim 11, wherein the signal processing chip comprises a front-end circuit configured to transform variation of the reluctivity of the magnetic sensors into a first electrical signal and a signal processing circuit configured to process the first electrical signal and output a second electrical signal representing a detection result of a DNA molecule to be detected.

14. The method for gene detection of claim 13, wherein the front-end circuit comprises a row address selector and a column address selector coordinated with each other and configured to allow the current to flow into selected magnetic sensors.

15. The method for gene detection of claim 14, wherein the front-end circuit further comprises a pre-amplifier configured to amplify the electrical signal representing variation of the reluctivity of the magnetic sensors so as to produce the first electrical signal.

16. The method for gene detection of claim 15, wherein the pre-amplifier comprises a differential amplifier, a first clipper stabilizing circuit, a second clipper stabilizing circuit, a third clipper stabilizing circuit, a first resistor and a second resistor; the differential amplifier comprises a positive input port, a negative input port, a first positive output port, a first negative output port, a second positive output port and a second negative output port; the first clipper stabilizing circuit is connected with the first positive output port and the first negative output port; the second clipper stabilizing circuit is connected with the second positive output port and the second negative output port; the third clipper stabilizing circuit is connected with the positive input port and the negative input port; the positive input port is connected with the first negative output port through the first resistor; the negative input port is connected with the second negative output port through the second resistor; the input signal of the differential amplifier is an electrical signal output from the magnetic sensors and representing variation of the reluctivity of the magnetic sensors, and input through the positive input port and the negative input port; the output signals of the differential amplifier comprise a first output signal output from the first positive output port and a second output signal output from the second positive output port.

17. The method for gene detection of claim 16, wherein the pre-amplifier further comprises a third resistor, a fourth resistor, a first capacitor and a second capacitor; the positive input port is connected with the ground through the third resistor and the first capacitor; the second negative output port is connected with the ground through the fourth resistor and the second capacitor.

18. The method for gene detection of claim 14, wherein the front-end circuit further comprises a biasing circuit; the biasing circuit comprises a plurality of diodes individually corresponding to the plurality of magnetic sensors; each diode is connected in series with a corresponding magnetic sensor so as to prevent the current from flowing into unselected magnetic sensors.
